(19) United States
(12) Patent Application Publication
(10) Pub. No.: US 2005/0255060 A1
(43) Pub. Date: Nov. 17, 2005

(54) Personal care compositions and methods regulating mammalian hair growth

(76) Inventors: John Erich Oblong, Loveland, OH (US); Sara Johnson McPhail, West Chester, OH (US); Shannon Christine Weitz, Hamilton, OH (US); Judith Lynn Harris, Cleves, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/125,385

(22) Filed: May 9, 2005

Related U.S. Application Data
(60) Provisional application No. 60/569,587, filed on May 10, 2004.

(51) Int. Cl. A61K 7/42; A61K 7/04; A61K 7/021; A61K 35/78

(52) U.S. Cl. 424/59; 424/61; 424/63; 424/401; 424/729

(57) ABSTRACT

Personal care compositions comprising at least one chronic skin care active selected from the group consisting of butylated hydroxytoluene (BHT), butylated hydroxyanisole (BHA), hexyl isobutyrate, menthyl anthranilate, methofuran, 3-butylidenepthalide, cetyl pyridinium chloride, green tea extract, catechins, phytosterols, ursolic acid, and plant extract compounds; at least one acute skin care active selected from the group consisting of particulate materials (including particles, pigments, and cross-linked silicone elastomers), alkane polyols, panthenol and pantothenic acid derivatives, and tanning actives; and a dermatologically acceptable carrier. The composition can also comprise the cationic thickener, Polyquaternium 37. The present invention also relates to methods of using such compositions to regulate hair growth and the condition of mammalian skin. Said methods generally comprise the step of topically applying the composition to the skin of a mammal needing such treatment, a safe and effective amount of such compositions.

### CROSS REFERENCE TO RELATED APPLICATION(S)

This application claims the benefit of U.S. Provisional Application No. 60/569,587, filed May 10, 2004.

### FIELD

The present invention relates to personal care compositions containing a combination of chronic skin care actives and acute skin care actives that are useful for regulating the condition of keratinous tissue, including, but not limited to, regulating mammalian hair growth.

### BACKGROUND

Various procedures and personal care products have been developed to remove unwanted hair, including shaving, electrolysis, depilatory creams or lotions, depilatory devices, waxing, plucking, therapeutic androgens, and laser hair removal. However, such conventional procedures frequently have drawbacks associated with them. Shaving, for instance, may cause nicks, cuts, rash and irritation and often leaves undesirable stubble. Electrolysis and laser hair removal can keep a treated area free of hair for prolonged periods of time, but can be either expensive, painful, and/or sometimes leave scarring. Waxing and plucking are painful and are poor options for shorter hair. Anti-androgens, used to treat female hirsutism, can have unpleasant physical side effects as well as possible birth defect implications. Since in non-hirsutistic females, the role of androgens is not required for normal hair growth, there is a need for technologies that do not function via androgen mediated pathways due to the birth defect implications. Depilatory devices can be painful to use and are not well-suited for many areas of the body including underarms and face. Finally, depilatory creams, although effective, are messy to apply and typically are not recommended for frequent use due to their high irritancy potential.

Therefore, a need exists for a safe, effective way to not only regulate the condition of mammalian keratinous tissue, but also to retard, inhibit, and/or stop unwanted mammalian hair growth on designated areas of the body.

### SUMMARY

The present invention relates to personal care compositions containing a combination of chronic skin care actives selected from the group consisting of butylated hydroxytoluene (BHT), butylated hydroxyanisole (BHA), hexyl isobutyrate, menthyl anthranilate, methofuran, 3-butylidenepthalide, cetyl pyridinium chloride, green tea extract, catechins, phytosterols, ursolic acid, and plant extract compounds in combination with acute skin care actives selected from the group consisting of particulate materials (including particles, pigments, and cross-linked silicone elastomers), alkane polyols, panthenol and pantothenic acid derivatives, and tanning actives. The personal care compositions can also include the cationic thickener, Polyquaternium 37. Such compositions are useful for regulating the condition of keratinous tissue, including, but not limited to, regulating mammalian hair growth.

The present invention also relates to methods of using such compositions to regulate hair growth and the condition of mammalian skin. These include such benefits as slower re-growth of the hair so the treated skin can be shaved less frequently, thereby reducing irritation and erythema, and wounding events such as nicks and cuts. By slowing down the re-growth of the hair, the hair becomes less noticeable, softer, and finer and the skin is left feeling smoother and silkier. Additional benefits include improvements in the ease of shaving and increased shaving efficiency. Said methods generally comprise the step of topically applying a safe and effective amount of such compositions to the skin of a mammal needing such treatment.

These and other features, aspects, and advantages of the present invention will become evident to those skilled in the art from a reading of the present disclosure.

### DETAILED DESCRIPTION

While the specification concludes with the claims particularly pointing and distinctly claiming the invention, it is believed that the present invention will be better understood from the following description.

Surprisingly, it has now been discovered that compositions of the present invention are useful for regulating mammalian hair growth, including retarding, inhibiting, or eliminating hair growth. Without being limited by theory, it is believed that the compositions of the present invention are able to modulate hair growth by inhibiting protease activity in and surrounding the hair follicular unit in mammalian skin. Proteases are key components in restructuring of the extracellular matrix during follicular progression through the dermis of skin in early anagen. Additionally, proteases play a role in angiogenesis, a key process for vascularization of the hair follicle during early anagen as well as maintenance of the vasculature bed during all of anagen.

Surprisingly, it has also been discovered that personal care compositions including butylated hydroxytoluene (BHT), butylated hydroxyanisole (BHA), hexyl isobutyrate, menthyl anthranilate, methofuran, 3-butylidenepthalide, cetyl pyridinium chloride, catechins, phytosterols, ursolic acid, and/or plant extract compounds can be used for regulating mammalian hair growth, including retarding, inhibiting, or eliminating hair growth.

All percentages, parts and ratios are based upon the total weight of the personal care compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “personal care compositions” are those used to treat or care for, or somehow moisturize, improve, or clean the skin and/or hair. Products contemplated by the phrase “personal care composition” include, but are not limited to, moisturizers, personal cleansing products, occlusive drug delivery patches, powders, wipes, hair conditioners, hair tonics, shampoos, hair colorants, skin treatment emulsions, shaving creams, antiperspirants, deodorants, and the like.

“Regulating hair growth,” namely mammalian hair growth, includes reducing, modulating, inhibiting, attenuating, retarding, promoting, enhancing, and/or the diminution of hair growth.

“Reduction/Inhibition of hair growth,” as referenced herein, is demonstrated when the frequency of hair removal is reduced, or the tactile and visual feel of mammalian hair is improved wherein the subject perceives less hair on the treated site (i.e., hair is perceived to be softer, finer, less noticeable), or quantitatively, when the weight of the hair removed by shaving (i.e., hair mass) is reduced thereby improving the ease, frequency, and effectiveness of shaving of a mammal.

“Mammalian hair,” as referenced herein, includes hair on any part of the body of a mammal and may include facial, cranial, or body hair. Male facial hair commonly refers to the beard, moustache, eyebrows and sideburns hair, but may include any area of the face and/or neck. Female facial hair (predominantly vellus but can include terminal hair) commonly refers to eyebrows, upper lip, chin, and cheeks area, but may also include any area of the face and/or neck. Other areas of hair growth typically desired to be regulated include underarms, bikini area, legs, arms, back, and chest. The desire for hair loss reduction is frequently associated with cranial hair, especially cranial hair in chemotherapy and/or radiation therapy patients.

The term “improving the tactile and visual feel,” as used herein, refers to the more noticeable improvement in the appearance of the hair on the skin such that it is perceived to be softer, finer, and/or less noticeable. Additionally, the ease, frequency, and effectiveness of shaving will be perceived by the mammal. Reduction of hair growth is demonstrated when the frequency of hair removal is reduced, or the subject perceives less hair on the treated site, or quantitatively, when the weight of hair removed by shaving (i.e., hair mass) is reduced.

The term “keratinous tissue,” as used herein, refers to keratin-containing layers disposed as the outermost protective covering of mammals (e.g., humans, dogs, cats, etc.) which includes, but is not limited to, skin, hair, etc.

The term “topical application,” as used herein, means to apply or spread the compositions of the present invention onto the surface of the keratinous tissue.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount,” as used herein, means an amount of a compound or composition sufficient to significantly induce a positive benefit, preferably a hair growth regulating benefit, or positive hair appearance or feel benefit, including independently or in combinations, the benefits disclosed herein, but low enough to avoid serious side effects, i.e., to provide a reasonable benefit to risk ratio, within the scope of sound judgment of the skilled artisan.

The term “ambient conditions,” as used herein, refers to surrounding conditions under about one atmosphere of pressure, at about 50% relative humidity, and at about 25° C., unless otherwise specified.

The compositions of the present invention are useful for regulating the growth of mammalian hair. Hair growth regulation is often desired in certain parts of the body due to hygiene or societal pressures. The desire for hair growth regulation may also stem from hair growth patterns induced or caused by factors internal and/or external to the body. Examples include environmental damage, radiation exposure (including ultraviolet radiation and radiation therapy), heredity, chronological aging, menopausal status (e.g., post-menopausal changes in hair growth), stress, diseases, chemotherapy, etc. The composition is particularly suitable for the treatment of hirsutism. In humans, the composition should be applied once or twice a day, or even more frequently, for at least three months to achieve a perceived reduction in hair growth.

The compositions of the present invention are stable. The ingredients used herein are compatible with each other and with the other skin care actives such as terpene alcohols, retinoids, peptides, tocopherol sorbate, and vitamin B₃ compounds. Additionally, the resulting skin care composition has good product stability and a reasonably long shelf-life.

The resulting compositions in combination with other selected skin care actives have good aesthetics. Examples of good aesthetics include compositions, such as luxurious creams and lotions, that (i) are light and nongreasy, (ii) have a smooth, silky feel upon the skin, (iii) spread easily, and/or (iv) absorb quickly. Other examples of good aesthetics include compositions that have a consumer acceptable appearance (i.e. no unpleasant odor or discoloration present), and provide good skin feel. The compositions herein may include a wide variety of other optional ingredients.

I. Chronic Skin Care Actives

A. Butylated Hydroxytoluene (BHT) and Butylated Hydroxyanisole (BHA)

The personal care compositions of the present invention may comprise a safe and effective amount of BHT or BHA. The BHT useful herein can be described by the general structure: 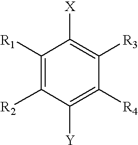
where in X is selected from the group consisting of OH and SH;
Y is selected from the group consisting of H, OH, OR₅, COOR₅, alkyl, cycloalkyl, heteroalkyl, heterocycloalkyl, aromatic, heteroaromatic, carboxamido, sulfonamido, carbamate, urea, and trialkylsilyl;
R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen;
R₅ is selected from the group consisting of alkyl, cycloalkyl, heteroalkyl, heterocycloalkyl, aromatic, heteroaromatic, trialkylsilyl, acyl, and hydrogen.

BHA and BHT can be purchased from various suppliers, including Eastman Chemical (Kingsport, Tenn.), Alfa Chemical (Kings Point, N.Y.), and Shell Chemical Company (Houston, Tex.).

BHT or BHA may be present in an amount of from about 0.0001% to about 50%, more preferably from about 0.001% to about 10%, more preferably from about 0.01% to about 5%, and even more preferably from about 0.1% to about 1.0% by weight of the composition.

B. Hexyl Isobutyrate

The personal care compositions of the present invention may comprise a safe and effective amount of hexyl isobutyrate, its salt, and derivatives thereof. Hexyl isobutyrate may be present in an amount of from about 0.0001% to about 50% by weight of the composition, more preferably from about 0.001% to about 10%, more preferably from about 0.01% to about 5%, and even more preferably from about 0.1% to about 2%.

C. Menthyl Anthranilate

The personal care compositions of the present invention may comprise a safe and effective amount of menthyl anthranilate, its salt, and derivatives thereof. Menthyl anthranilate is commercially available from Phoenix Aromas & Essential Oils, Inc. (Norwood, N.J.) and Alzo International (Sayreville, N.J.).

Menthyl anthranilate may be present in an amount of from about 0.0001% to about 50% by weight of the composition, more preferably from about 0.001% to about 20%, more preferably from about 0.01% to about 10%, and even more preferably from about 0.1% to about 5%.

D. Methofuran

The personal care compositions of the present invention may comprise a safe and effective amount of methofuran, its salt, and derivatives thereof. Methofuran is commercially available from Aldrich Chemical Company (Milwaukee, Wis.), and Sigma Chemical Company (St. Louis, Mo.).

Methofuran may be present in an amount of from about 0.0001% to about 50% by weight of the composition, more preferably from about 0.001% to about 20%, more preferably from about 0.01% to about 10%, and even more preferably from about 0.1% to about 5%.

E. 3-Butylidenepthalide

The personal care compositions of the present invention may comprise a safe and effective amount of 3-butylidenepthalide, its salt, and derivatives thereof. 3-butylidenepthalide may be present in an amount of from about 0.0001% to about 50% by weight of the composition, more preferably from about 0.001% to about 20%, more preferably from about 0.01% to about 10%, and even more preferably from about 0.1% to about 5%.

F. Cetyl Pyridinium Chloride

The personal care compositions of the present invention may comprise a safe and effective amount of cetyl pyridinium chloride. Alternate forms of cetyl pyridinium chloride include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215.

Cetyl pyridinium chloride may be present in an amount of from about 0.0001% to about 50% by weight of the composition, more preferably from about 0.001% to about 5%, more preferably from about 0.01% to about 2%, and even more preferably from about 0.05% to about 1%.

G. Green Tea Extract and Catechins

The personal care compositions of the present invention may comprise a safe and effective amount of one or more catechin compounds selected from the group consisting of green tea extracts, catechin, epicatechin, epigallocatechin, epicatechin gallate, epigallocatechin gallate, gallocatechin, and mixtures thereof. Preferably, the catechin is free of caffeine and is extracted and enriched from a green tea plant source. More preferably, the catechin is epigallocatechin gallate. Various purified forms of green tea extracts and catechins are commercially available from Sabinsa (Piscataway, N.J.), Active Organics (Lewisville, Tex.), and Arch Personal Care Products (South Plainfield, N.J.). Ideally, the green tea extract is colorless and devoid of tannins and other color impurities.

The catechin mixture may be present in an amount of from about 0.0001% to about 50% by weight of the composition, more preferably from about 0.001% to about 10%, more preferably from about 0.01% to about 5%, and even more preferably from about 0.1% to about 2.5%.

H. Phytosterols

The personal care compositions of the present invention may comprise a safe and effective amount of one or more phytosterols selected from the group consisting of β-sitosterol, campesterol, brassicasterol, Δ5-avennasterol, lupenol, α-spinasterol, stigmasterol, their derivatives, and combinations thereof. More preferably, the phytosterol is selected from the group consisting of β-sitosterol, campesterol, brassicasterol, stigmasterol, their derivatives, and combinations thereof.

Phytosterols of the present invention can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers, tautomers, and derivatives (e.g., esters) of such and are commercially available from Aldrich Chemical Company (Milwaukee, Wis.), Sigma Chemical Company (St. Louis, Mo.), Cognis, and Karlshamns (Karlshamns, Sweden).

Phytosterol may be present in an amount of from about 0.01% to about 50%, by weight of the composition, more preferably from about 0.1% to about 20%, more preferably from about 0.2% to about 15%, and even more preferably from about 0.5% to about 10%.

I. Ursolic Acid

The personal care compositions of the present invention may comprise a safe and effective amount of ursolic acid. Ursolic acid of the present invention can be synthetic or natural in origin and can be used as essentially pure compounds or mixtures of compounds (e.g., extracts from natural sources). Ursolic acid is commercially available from such suppliers as Sabinsa (Piscataway, N.J.) and Crodarom S. A. S. (Chanac, France). Ursolic acid may be present in an amount of from about 0.0001% to about 50% by weight of the composition, more preferably from about 0.001% to about 10%, more preferably from about 0.01% to about 7.5%, and even more preferably from about 0.05% to about 5%.

J. Compounds Derived from Plant Extracts

The personal care compositions of the present invention may comprise a safe and effective amount of compounds derived from plant extracts selected from the group consisting of leguminosae, solanaceae, gramineae, and cucurbitaceae. Preferably, the compound derived from plant extracts is a protease inhibitor and one or more isoflavones. Isoflavone examples include genistein and daidzein. Compounds derived from plant extracts may be present in an amount of from about 0.0001% to about 50% by weight of the composition, more preferably from about 0.001% to about 10%, even more preferably from about 0.01% to about 7.5%, and still more preferably from about 0.05% to about 5%.

II. Acute Skin Care Actives

A. Particulate Materials

The compositions of the present invention may comprise one or more particulate materials. Nonlimiting examples of particulate materials useful in the present invention include particles, pigments, and cross-linked silicone elastomers.

Particulate materials useful herein include colored and uncolored pigments, interference pigments, inorganic powders, organic powders, composite powders, optical brightener particles, exfoliants and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. These particulate materials may provide a wide range of functions, including but not limited to modifying skin feel, masking the appearance of certain skin characteristics such as blotchy areas, age spots, freckles, fine lines, wrinkles, and pores, absorbing excess skin sebum/oils, reducing skin shine, improving application properties of the composition, masking the color of other components of the composition, filling in skin pores, lines and wrinkles, and reducing migration of liquid materials on the skin. If used, particulate materials are present in the composition in levels of from about 0.01% to about 20%, more preferably from about 0.05% to about 10%, still more preferably from about 0.1% to about 5%, by weight of the composition. There are no specific limitations as to the pigment, colorant or filler powders used in the composition.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminum starch octenyl succinate, silk, glass, fibers, ground seeds, pumice, and mixtures thereof. Especially preferred are spherical powders with an average primary particle size from about 0.1 to about 75 microns, preferably from about 0.2 to about 30 microns.

Suitable organopolysiloxane gel compositions are dimethicone/vinyl dimethicone crosspolymers swollen in an appropriate solvent. Such dimethicone/vinyl dimethicone crosspolymers are supplied by a variety of suppliers including Dow Corning (DC 9040™ and DC 9041™), General Electric (SFE 839™), Shin Etsu (KSG-15™, KSG-16™, KSG-18™ [dimethicone/phenyl vinyl dimethicone crosspolymer]) and lauryl dimethicone/vinyl dimethicone crosspolymers supplied by Shin Etsu (e.g., KSG-31™, KSG-32™, KSG-41™, KSG-42™, KSG-43™, and KSG-44™). Alternatively, organopolysiloxane elastomer powders can be used, suitable examples include vinyl dimethicone/methicone silesquioxane crosspolymers like Shin-Etsu's KSP-100™, KSP-101™, KSP-102™, KSP-103™, KSP-104™, KSP-105™, hybrid silicone powders that contain a fluoroalkyl group like Shin-Etsu's KSP-200™, and hybrid silicone powders that contain a phenyl group such as Shin-Etsu's KSP-300™; and Dow Corning's DC 9506™.

Also useful herein are interference pigments. Interference pigments, for purposes of the present invention are defined as thin platelike layered particles having two or more layers of controlled thickness with different refractive indices that yield a characteristic reflected color from the interference of typically two, but occasionally more, light reflections, from different layers of the platelike particle. Examples of interference pigments are micas layered with about 50-300 nm films of TiO2, Fe2O3, silica, tin oxide, and/or Cr2O3. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™), Eckart (e.g. Prestige and Prestige Silk lines). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887.

The pigments/powders useful herein can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobically treatments being preferred. Particularly useful hydrophobic pigment treatments include polysiloxane treatments such as those disclosed in U.S. Pat. No. 5,143,722.

B. Alkane Polyols

Incorporation of the alkane polyols in the personal care compositions increase synergistically the efficacy of the hair growth inhibitors. Without wishing to be bound by theory, it is believed that this may be due in part to the hair growth inhibition activity of the alkane polyols themselves, and/or the ability of the alkane polyols to potentiate the delivery of other hair growth inhibitors to the hair follicle via either transdermal penetration or a shunt route.

Preferred alkane polyols for use herein include C6 to C9 alkane polyols, or mixtures thereof, more preferably C6 to C9 alkane diols, or mixtures thereof. Non-limiting examples of alkane polyols suitable for use herein include 1,2-hexanediol, 2-ethyl-1,3-hexanediol, 1,2-heptanediol, 1,7-heptanediol, 1,2-octanediol, 1,8-octanediol, 1,9-nonanediol, or mixtures thereof. More preferably still, the alkane polyol herein comprises C₆-C₈ alkane diols or mixtures thereof, non-limiting examples of which include 1,2-hexanediol, 2-ethyl-1,3-hexanediol, 1,2-heptanediol, 1,7-heptanediol, 1,2-octanediol, 1,8-octanediol, or mixtures thereof, preferably 1,2-hexanediol.

The compositions of the present invention preferably comprise from about 0.01% to 20% of an alkane polyol, more preferably from 0.05% to 10%, more preferably still from 0.5% to 3%, by weight of the composition.

C. Panthenol and Pantothenic Acid Derivatives

The topical leave-on compositions of the present invention may comprise panthenol or pantothenic acid derivatives.

The panthenol and its derivatives include D-panthenol ([R]-2,4-dihydroxy-N-[3-hydroxypropyl)]-3,3-dimethylbutamide), DL-panthenol, pantothenic acids and their salts, preferably the calcium salt, panthenyl triacetate, royal jelly, panthetine, pantotheine, panthenyl ethyl ether, pangamic acid, pantoyl lactose, Vitamin B complex, or mixtures thereof. The compositions of this invention may comprise a safe and effective amount of the panthenol or its derivative, such that the resultant composition is safe and effective for regulating skin texture without excessive stickiness. The panthenol derivative is preferably used in an amount of from about 0.01% to 10%, more preferably from 0.1% to about 5%, more preferably still from about 0.2% to about 3%.

Compositions comprising pantothenic acid derivatives that remain more stable than panthenol and other similar materials in acidic compositions or in compositions containing acid-producing materials such as aluminum-containing actives, and are also suitable for application to the skin. The selected pantothenic acid derivatives are most typically in liquid form and dispersed throughout or otherwise solubilized within the liquid carrier component of the composition.

The term “pantothenic acid derivative” as used herein refers to those materials that conform to the formula: 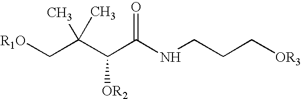
wherein R₁, R₂ and R₃ are hydrogen, C2-C20 hydrocarbons, C2-C20 carboxylic acid esters, or combinations thereof, provided that not more than two of R₁, R₂ and R₃ are hydrogen. Preferably, R₁, R₂ and R₃ are independently selected from hydrogen, C2-C8 hydrocarbons, C2-C8 carboxylic acid esters, or combinations thereof; more preferably, R₁ and R₂ are hydrogen, and R₃ is a C2-C8 hydrocarbon, C2-C8 carboxylic acid ester, or combinations thereof; even more preferably, R₁ and R₂ are hydrogen and R₃ is ethyl. The selected pantothenic acid derivatives may be derived or otherwise obtained from any known source, which may include pantothenic acid or materials other than pantothenic acid, so long as the resulting material has the above defined chemical formula.

Specific non-limiting examples of selected pantothenic acid derivatives for use herein include ethyl panthenol, panthenyl triacetate, and combinations thereof. Preferred are the d-isomeric forms of such derivative forms, most preferably d-ethyl panthenol.

The concentration of the pantothenic acid derivative for use in the compositions of the present invention preferably ranges from about 0.01% to about 10%, more preferably from about 0.05% to about 5%, even more preferably from about 0.5% to about 3%, by weight of the composition.

D. Tanning Actives

The personal care compositions of the present invention may comprise a self-tanning agent. As used herein, the term “self-tanning agent” includes α-hydroxy aldehydes and ketones such as dihydroxyacetone and structurally related compounds. This definition includes all such agents that are similarly useful in producing or inducing the artificial tanning process in human skin. Accordingly, the compositions of the present invention comprise an α-hydroxy aldehyde or ketone of the formula (I): 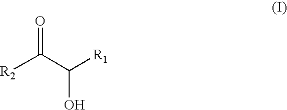
wherein R₁ is H, CH₂OH, CHOHCH₂OH, CH(OH)CH(═O), CH(OCH₃)CH(═O), CH(NH₂)CH(═O), or CH(NH-Phenyl)CH(═O); and R₂ is H or CH₂OH. Dihydroxyacetone (DHA) itself may be represented by the following general structural formula:

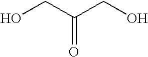

Other compounds useful herein include Glyceraldehyde, 2,3-dihydroxy-succindialdehyde, 2,3-Dimethoxysuccindialdehyde, Erythrulose, Erythrose, 2-Amino-3-hydroxy-succindialdehyde, 2-Benzylamino-3-hydroxy-succindialdehyde. Preferably, the self-tanning agent comprises DHA, erythrulose, or mixtures thereof, more preferably DHA. Preferably the compositions of the present invention comprise from about 0.1% to about 10% of the self-tanning agent. More preferably, the compositions of the present invention comprise from about 1% to about 6%, of the self tanning agent.

III. Dermatologically Acceptable Carrier

The personal care compositions of the present invention also comprise a dermatologically acceptable carrier. The phrase “dermatologically acceptable carrier”, as used herein, means that the carrier is suitable for topical application to the keratinous tissue, has good aesthetic properties, is compatible with the actives of the present invention and any other components, and will not cause any safety or toxicity concerns. The dermatologically acceptable carrier may be present in an amount of from about 50% to about 99.99%, preferably from about 60% to about 99.95%, more preferably from about 70% to about 98%, and even more preferably from about 80% to about 95% by weight of the composition.

The carrier can be in a wide variety of forms. For example, emulsion carriers, including, but not limited to, oil-in-water, water-in-oil, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in-silicone emulsions. As will be understood by the skilled artisan, a given component will distribute primarily into either the water or oil phase, depending on the water solubility/dispensability of the component in the composition. The catechin compound distributes primarily into the oil phase. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 1% to about 10%, more preferably from about 2% to about 5%, of an emulsifier, based on the weight of the composition. Emulsifiers may be nonionic, anionic or cationic.

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form. Preferred water-in-silicone and oil-in-water emulsions are described in greater detail below.

A. Water-In-Silicone Emulsion

Water-in-silicone emulsions contain a continuous silicone phase and a dispersed aqueous phase.

1. Continuous Silicone Phase

Preferred water-in-silicone emulsions of the present invention may comprise from about 1% to about 60%, preferably from about 5% to about 40%, more preferably from about 10% to about 20%, by weight of a continuous silicone phase. The continuous silicone phase exists as an external phase that contains or surrounds the discontinuous aqueous phase described hereinafter.

The continuous silicone phase contains an organopolysiloxane oil. The organopolysiloxane oil for use in the composition may be volatile, non-volatile, or a mixture of volatile and non-volatile silicones.

Also useful are materials such as polyalkylsiloxanes, cyclic polyalkylsiloxanes, trimethylsiloxysilicate, dimethiconols, polyalkylaryl siloxanes are also suitable for use in the composition.

Preferred for use herein are organopolysiloxanes selected from the group consisting of polyalkylsiloxanes, alkyl substituted dimethicones, cyclomethicones, trimethylsiloxysilicates, dimethiconols, polyalkylaryl siloxanes, and mixtures thereof.

2. Dispersed Aqueous Phase

The personal care compositions of the present invention may comprise from about 30% to about 90%, more preferably from about 50% to about 85%, and even more preferably from about 70% to about 80% of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore.

The aqueous phase can be water, or a combination of water and one or more water soluble or dispersible ingredients. Nonlimiting examples of such optional ingredients include thickeners, acids, bases, salts, chelants, gums, water-soluble or dispersible alcohols and polyols, buffers, preservatives, sunscreening agents, colorings, and the like.

3. Emulsifier for Dispersing the Aqueous Phase

The water-in-silicone emulsions of the present invention preferably comprise an emulsifier. A wide variety of emulsifying agents can be employed herein to form the preferred water-in-silicone emulsion. Known or conventional emulsifying agents can be used in the composition, provided that the selected emulsifying agent is chemically and physically compatible with essential components of the composition, and provides the desired dispersion characteristics. These silicone emulsifiers are typically organically modified organopolysiloxanes. Useful silicone emulsifiers include dimethicone copolyols. Other examples include alkyl-modified dimethicone copolyols, i.e., compounds that contain C2-C30 pendant side chains. Still other useful dimethicone copolyols include materials having various cationic, anionic, amphoteric, and zwitterionic pendant moieties.

Among the non-silicone-containing emulsifiers useful herein are various non-ionic and anionic emulsifying agents.

B. Oil-In-Water Emulsions

Other preferred topical carriers include oil-in-water emulsions, having a continuous aqueous phase and a hydrophobic, water-insoluble phase (“oil phase”) dispersed therein. An especially preferred oil-in-water emulsion, containing a structuring agent, hydrophilic surfactant and water, is described in detail hereinafter.

1. Structuring Agent

A preferred oil-in-water emulsion comprises a structuring agent to assist in the formation of a liquid crystalline gel network structure. Without being limited by theory, it is believed that the structuring agent assists in providing rheological characteristics to the composition that contribute to the stability of the composition. The structuring agent may also function as an emulsifier or surfactant.

The preferred structuring agents of the present invention are selected from the group consisting of stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

2. Hydrophilic Surfactant

The preferred oil-in-water emulsions comprise from about 0.05% to about 10%, preferably from about 1% to about 6%, and more preferably from about 1% to about 3% of at least one hydrophilic surfactant which can disperse the hydrophobic materials in the water phase (percentages by weight of the composition). The surfactant, at a minimum, must be hydrophilic enough to disperse in water.

Suitable surfactants include any of a wide variety of known cationic, anionic, zwitterionic, amphoteric, and nonionic surfactants.

3. Water

The preferred oil-in-water emulsion comprises from about 25% to about 98%, preferably from about 65% to about 95%, more preferably from about 70% to about 90% water by weight of the topical carrier.

4. Composition Forms

The personal care compositions of the present invention, including but not limited to lotions and creams, may comprise a dermatologically acceptable emollient. Such compositions preferably contain from about 2% to about 50% of the emollient. A preferred emollient is glycerin.

Ointments of the present invention may comprise a simple carrier base of animal or vegetable oils or semi-solid hydrocarbons (oleaginous); absorption ointment bases which absorb water to form emulsions; or water soluble carriers, e.g., a water soluble solution carrier. Ointments may further comprise a thickening agent.

Compositions of this invention useful for cleansing (“cleansers”) are formulated with a suitable carrier, and preferably contain from about 1% to about 90% of a dermatologically acceptable surfactant.

The compositions of the present invention may also be in the form of cosmetics. Suitable cosmetic forms include, but are not limited to, foundations, lipsticks, rouges, mascaras, and the like.

IV. Optional Components

The compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

A. Hair Growth Inhibiting Compounds

The compositions of the present invention may also comprise other hair growth inhibiting compounds. These compounds are known in the art as inhibiting hair growth and consist of the following classes and examples.

1. Natural Plant Extracts

Natural plant extracts useful herein include compounds extracted from any part of the plant of saw palmetto, willow herb, pumpkin seed, creosote, sea-buckthorn oil, capsicum, Echinacea angustifolia, Echinacea purpurea, Lithosperumum, Rosaceae, Sanguisorba officinalis, Tropaeolum majus, white birch and rubiaceae plant groups, Juniperus genus, malt, from genus Centipeda, Cinnamonum verum, Curcurbita pepo, Epilobium roseum, Salvia officinalis, Cassia obtusifoila Linne, Pleione genus, Curcuma longa, Salix alba, Hamamelis virginiana, Diopyros kaki, Hydrangea macrophylla, Hydrangea serrata, Iridaceae genus, Moraceae Humulus, Ikurinin, Regulo plant (Abelmoschus moschatus), Wolo plant (Borassus flabellifer), Hedera helix, Lithospermu, Scutellaria genus, tomato, Commiphora myrrha, Cymbopogon nardus, Lagerstroemia speciosa, Phyllanthus nuriri, Smilax zeylanica, Woodfordia fruticosa, Cistanche salsa, Larrea divaricata, Plantago asiatica, Stachys sieboldii, lavender, lemon, carrot, ginger, clove, honey, juniper, almond, palmarosa, eucalyptus, rosemary, sugars, Coix lachryma-jobi, bur marigold infusion, bacterium ribosomes, conifer extract, daisy infusion, tea tree oil, spearmint, honey, ant eggs, Bowman Birk inhibitor, peach oil, essential oils, aloe, elasatin decomposition enzyme inhibitor, peptides, plant fruit enzymes, shogaol, zingerone, zingiberol, and zingiberone

2. Metabolic Modulators

Metabolic modulators useful herein include 5′-p-fluorosulphonyl benzoyl adenosine, 5-keto-D-fructose, 5-keto-D-fructose-1,6-bisphosphate, 6-amino-6-deoxy-glucose, inhibitor of a cysteine pathway enzyme, guanidino succinic acid, cysteine sulphinic acid, phosphoglycerate, cysteamine, cysteine sulphinic acid, cysteinyl-glycine, D-cysteine, inhibitor of a cholesterol pathway enzyme, inhibitor of the formation of glycoproteins, N-acetylcysteine (NAC), D-mannosamine, N-alpha-(p-tosyl)-L-lysine chloromethyl ketone, N-acetyl-beta-D-mannosamine, oxaloacetic acid, finasteride, arginase inhibitor, N-phosphonoacetyl-aspartic acid, N-alpha-acetyl-L-arginine, N-alpha-benzoyl-L-argininamide, N-alpha-benzoyl-L-arginine, N-alpha-benzoyl-L-arginine methyl ester, NG-L-arginine benzyl ester, NG-nitro-L-arginine, NG-nitro-L-arginine methyl ester, dithiothreitol, glutathione, homocysteine, lipoic acid, 2-mercaptoethanol, mecaptopropionic acid, thiodiglycol, thiodiglycolic acid, thioglycerol, thioglycolic acid, thiolactic acid, thiomalic acid, thiopropionic acid, thiosalicylic acid, thioxanthine, H-homoarginine, L-alanosine, L-argininamide, L-asparaginamide, L-cysteine methyl ester, alpha-methyl-DL-methionine, dimethyl cysteamine, sulfotransferase inhibitors, N(G)-methyl-L-arginine, alpha.-fluoromethylhistidine, inhibitors of glutamine metabolism, glutathione synthesis stimulators, fatty acids, chelating agents, pravastatin, rivastatin, simvastatin, squalestatin, fluvastatin, mevastatin, mevinolin, lovastatin, cysteine, chlorotaurine, 2-mercaptopropionic acid, diethyldithiocarbamic acid, aromatase inhibitors, glutathione S-transferase modulators, peptide or trisamine carrying fatty acid ester and dithioalkanoyl groups, sorbic acid, vitamin K, vitamin F, and phloretin.

3. Anti-Proliferatives

Anti-proliferatives useful herein include ifluoromethylomithine (DFMO), methacycline, protein kinase C inhibitors, protein-tyrosine kinase inhibitors, tyrphostins and tryphostins, cyclooxygenase inhibitors, 5-alpha-reductase inhibitors, adenylsuccinate synthetase inhibitor, aspartate transcarbamylase inhibitor, gammaglutamyl transpeptidase inhibitor, ornithine decarboxylase inhibitors, non-steroidal anti-inflammatory drugs (NSAIDS), lipoxygenase inhibitors and stimulants, nordihydroguaianetic acid (NDGA), inhibitor of alkaline phosphatase, doxycycline, minocycline, taxodione, taxodone, bacteriostatic or haemostyptic agent, especially stannous fluoride, alpha-ethyl-ornithine, nalidixic acid, tetracycline, inhibitors of the hypusine biosynthetic pathway, methacycline, methylglyoxal bis(guanylhydrazone), bromocryptine, trihydroxypurine, etoposide, guanidino succinic acid, matlystatin-B, 5′-deoxy-5′-(N-methyl-N-(2-aminooxyethyl)-aminoadenosine (MAOEA), 5′-deoxy-5′-methyl-thioadenosine, doxycycline, pyrimidine-cyanoguanidine derivatives, substituted amidine or guanidine, butyric acid derivatives, hydroxamic acid and analogues, medroxyprogesterone acetate, megestrol acetate, melengestrol acetate, nomegestrol acetate, mycophenolic acid, cyanoguanidine derivatives, and diethyl glyoxal bis(quanylhydrazone).

4. Signal Transduction Modulators

Signal transduction modfulators useful herein include Br-cAMP, E6AP-binding polypeptides, ethoxyquin, anti-angiogenic steroids, CDK binding proteins, chimeric polypeptide with cyclin-dependent kinase (CDK) binding motif, suppressor of angiogenesis, alpha- or gamma-linolenic acid, EGF and analogues, Hairless protein and analogues, estrogen agonists or antagonists, proteoglycans or glycosaminoglycans, phytoestrogen, hedgehog antagonists, patched antagonists, interleukin-1 antagonist, alpha-TNF antagonist, leuteinizing hormone-releasing hormone and analogues, GnRH inhibitors, Heptapeptide luteinizing hormone releasing hormone (LHRH) analogs, 1-halomethyl-5alpha-androstanes and delta-androstenes, 3-oxo-4-aza-5 alpha-androstane derivatives, finasteride, spironolactone, propyl gallate, eicosapentaenoic acid, lavendustin A, activin A, androgen receptor blockers, quercetin, protocatechuic acid and aldehyde, methyl caffeate, apigenin, caffeic acid, progestins and antiprogestins, vitamin D and analogues including previtamin D and provitamin D, androstenedione analogues, lipoxydase, spironolactone, cyproterone acetate, progesterone, and melatonin

5. Proteases and Protease Inhibitors

Protease and protease inhibitors useful herein include 1,10-phenanthroline, elastase inhibitors, papain, trypsin and analogues, chymotrypsin, pepsin, bromelain, ficin, pancreatin, and marimistat.

6. Others

Other hair growth inhibiting compounds useful herein include phlondrin, agaric acid, vernolepin, D-penicillamine, ethacrynic acid, eupacunin, euparotin acetate, diethylaminomalonate, protocatechuic aldehyde, non-elastomeric polyolefin resin, partially fluorinated polyolefin resin, quinaldic acid, 1,8-diaminooctane, 2-methyl-6-heptyne-2,5-diamine, 3-carboxypropyl disulphide, 5-(N-benzyloxycarbonyl)-1-phenylalanamidomethyl)-3-bromo-4,5-dihydroisoxazole, 6-heptyne-2,4-diamine, actinonin, batimistat, captopril, diethyl aminomalonate, diethyldithiocarbamic acid, estramustine, ethacrynic acid, meso-dimercaptosuccinic acid, N-[N[((R)-1-phosphonopropyl)-(S)-leucyl]-(S)-phenylalanine-N-methylamide, N-phosphonalkyl dipeptides, oxaloacetic acid, phosphocysteamine, S-carbamyl-L-cysteine, S-trityl-L-cysteine, sulphasalazine, thiosalicylic acid, tyramine, 2-difluoromethyl-, 2,5-diamino pentanoic acid, herbimycin, HNMPA (AM)3, O-p-nitrohydroxylamine, cromoglycate, quinoline-3-carboxamide, 16 alpha- or beta-substituted 4-aza-5 alpha-androst-1-en-3-ones, 2-aryl-indole derivatives, 2-phenyl-3-aminoalkyl-indole derivatives, 5 alpha-androstan-3-ones, 5-(aminocarbonylalkyl)-3-(heterobicyclyl-alkylaminoalkyl)-2-phenylindole derivatives, 6-azaindole derivatives, 7-azaindole derivatives, aryl-imidazo-pyridines, carboxyalkylamine derivatives, malonamide derivatives, 2-indole carboxylic acid derivatives, aminopropanes, diethylenediamines, histamine antagonist, phenothiazines, tetrazolyl-benzofuran carboxamides, tetrazolyl-benzothiophene carboxamides, 17alpha-hydroxy-4,9(11)-pregnadiene-3,20-dione derivatives, benzothiophene derivatives, (−)cis 6(S)-phenyl-5(R)-[4-(2-pyrrolidin-1-ylethoxyphenyl]-5,6,7,8-tetrahydronaphthelen-2-ol D-tartrate (I), tetrahydronaphthalene derivatives, tetrahydroisoquinolines, tetrahydroisoquinoline derivatives, tetrahydroisoquinoline derivatives, 3-(anilinomethylene)oxindole derivatives, benzo-[f]-quinolin-3-one derivative, ((S-(−)-N-(alpha-ethylbenzyl)-3-hydroxy-2-phenylquinoline-4-carboxamide), 24-ethyl-(delta)4,22-cholestadien-3-one, benzoic acid lactone ether, copper, iron, zinc, 1 dehydromelengestrol acetate, 1-dehydromegestrol acetate, chlormadinone acetate, cyproterone acetate, hydrindanes, diazo compounds, tetrahydroisoquinolines, tetrahydronaphthalenes, 3-amino-2,3-dihydro benzoic acid, 6-fluoro-2,5-diamino hexanoic acid, (S)-2-amino-4-amino oxy-butyric acid, triarylmethane compounds, perfluoro-substituted aniline derivatives, 17alpha-propyltestosterone, 4-androstene-3-one 17beta-carboxylic acid, (4R)-5,10-seco-19-norpregna 4,5-diene-3,10,20-trione, chlormadinone acetate, 2-substituted 6-tetra hydronaphthyl or indanyl naphthalene derives, 2-phenyl-benzothiophene derivatives, 2-arylimino-oxaza or thiaza heterocyclic compounds, indole derivatives, dormant cell extracts, N-substituted benzyl- or thienylmethyl-4-pyridone compounds, (1H)-benzo(c)quinolizin-3-one derivatives, citric acid, Dead Sea salts, visaborol, chlorophenol, o-phenylphenol, phenol, niphtolide, 2-amino-5-substituted benzophenone, aniline derivatives, camphor oil, citric acid, conjugate comprising active agent substituted with amino acid, 11-beta-aryl-17-spiro-pyrrolin-2-ylidene N-oxide steroid, phenyl imidazolidines, coumarin derivatives, borneol, cineole, linalool, methyl heptenone, thiomolybdate compound, and trifluoroanilide derivatives.

B. Polyquaternium-37

The compositions of the present invention may also comprise the synthetic cationic polymer Polyquaternium-37 (methacryloylethyl trimethyl ammonium chloride homopolymer). This polymer may be added to the composition as a powder or as a liquid dispersion. This polymer is commercially available under the tradenames Synthalen (3V Sigma), Ultragel 300 (Cosmetic Rheologies Ltd), Rheocare CTH(E) (Cosmetic Rheologies Ltd.), Salcare SC95 and Salcare SC96 (Ciba Specialty Chemicals).

C. Depilatories

Certain embodiments of the present invention may optionally contain a depilatory. As used herein, “depilatory” means an agent capable of removing hair from the skin by cleaving the disulfide bonds in hair keratin, thereby causing the hair fiber to disintegrate. Preferred depilatories useful in the subject invention include ammonium thioglycolate, barium sulfate, calcium thioglycolate, ethanolamine thioglycolate, potassium thioglycolate, sodium thioglycolate, thioglycolic acid and thioacetic acid. Examples of suitable depilatories are described in further detail in U.S. Pat. No. 5,897,857.

D. Desquamation Actives

A safe and effective amount of a desquamation active may be added to the compositions of the present invention. One desquamation system that is suitable for use herein comprises sulfhydryl compounds, salicylic acid, and zwitterionic surfactants.

E. Anti-Acne Actives

The compositions of the present invention may comprise a safe and effective amount of one or more anti-acne actives. Examples of useful anti-acne actives include resorcinol, sulfur, salicylic acid, erythromycin, zinc, etc.

F. Anti-Wrinkle Actives/Anti-Atrophy Actives

The compositions of the present invention may further comprise a safe and effective amount of one or more anti-wrinkle actives or anti-atrophy actives. Exemplary anti-wrinkle/anti-atrophy actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., salicylic acid, glycolic acid), keto acids (e.g., pyruvic acid), ascorbic acid (vitamin C), phytic acid, lipoic acid; lysophosphatidic acid, skin peel agents (e.g., phenol and the like), flavonoids (e.g., flavanones, chalcones, isoflavones, flavones, etc.), boswellic acid, stilbenes, cinnamates, resveratrol, kinetin, zeatin, dimethylaminoethanol, peptides from natural sources (e.g., soy peptides), salts of sugar acids (e.g., Mn gluconate), Coenzyme Q₁₀ (ubiquinone, vitamin Q₁₀), terpene alcohols (e.g., farnesol), peptides, vitamin B₃ compounds and retinoids, and other vitamin B compounds (e.g., thiamine (vitamin B1), pantothenic acid (vitamin B5), carnitine (vitamin Bt), riboflavin (vitamin B2), cobalamine (vitamin B12), pangamic acid or diisopropylamine dichloroacetate (vitamin B15's), and their derivatives and salts (e.g., HCl salts or calcium salts)).

1. Vitamin B₃ Compounds

The compositions of the present invention may comprise a safe and effective amount of a vitamin B₃ compound. Vitamin B₃ compounds are particularly useful for regulating skin conditions. Vitamin B₃ compounds may be present in an amount of from about 0.01% to about 50%, more preferably from about 0.1% to about 10%, even more preferably from about 0.5% to about 10%, and still more preferably from about 1% to about 5%, even more preferably from about 2% to about 5%, by weight of the composition.

2. Retinoids

The compositions of the present invention may also comprise a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters (saturated or unsaturated alkyl chains) of retinol, including retinyl palmitate, retinyl acetate, retinyl propionate), retinal, and/or retinoic acid (including all-trans retinoic acid and/or 13-cis-retinoic acid), more preferably retinoids other than retinoic acid.

G. Additional Optional Components

1. Anti-Oxidants/Radical Scavengers

The compositions of the present invention may include a safe and effective amount of an anti-oxidant/radical scavenger. The anti-oxidant/radical scavenger is especially useful for providing protection against UV radiation that can cause increased scaling or texture changes in the stratum corneum and against other environmental agents, which can cause skin damage.

Anti-oxidants/radical scavengers such as ascorbic acid (vitamin C) and its salts, ascorbyl esters of fatty acids, ascorbic acid derivatives (e.g., magnesium ascorbyl phosphate, ascorbyl glucoside), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate.

2. Chelators

The compositions of the present invention may comprise a safe and effective amount of a chelator or chelating agent. As used herein, “chelator” or “chelating agent” means an active agent capable of removing a metal ion from a system by forming a complex so that the metal ion cannot readily participate in or catalyze chemical reactions.

3. Anti-Inflammatory Agents

A safe and effective amount of an anti-inflammatory agent may be added to the compositions of the present invention, preferably from about 0.1% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency. Anti-inflammatories can be selected from several classes. One is comprised of steroidal anti-inflammatory agents, including but not limited to, corticosteroids. The preferred steroidal anti-inflammatory for use is hydrocortisone.

A second class of anti-inflammatory agents, which is useful in the compositions, includes the nonsteroidal anti-inflammatory agents. Such compounds are known in the art as non-steroidal anti-inflammatory agents (“NSAIDS”) and are described in detail, along with methods for manufacture in the following U.S. patents: U.S. Pat. Nos. 5,280,045; 4,708,966; 5,189,066; 5,510,361; 5,189,066; 5,476,876; and 5,684,204.

Mixtures of these non-steroidal anti-inflammatory agents may also be employed, as well as the dermatologically acceptable salts and esters of these agents.

Finally, so-called “natural” anti-inflammatory agents are useful in methods of the present invention. Such agents may suitably be obtained as an extract by suitable physical and/or chemical isolation from natural sources (e.g., plants, fungi, and by-products of microorganisms).

Additional anti-inflammatory agents useful herein include allantoin and compounds of the Licorice (the plant genus/species Glycyrrhiza glabra) family, including glycyrrhetic acid, glycyrrhizic acid, and derivatives (e.g., salts and esters). Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

The active component of these anti-inflammatory agents (e.g., bisabolol, glycyrrhetinate esters) may also be obtained via extraction from natural sources or prepared synthetically.

4. Anti-Cellulite Agents

The compositions of the present invention may comprise a safe and effective amount of an anti-cellulite agent. Suitable agents may include, but are not limited to, xanthine compounds (e.g., caffeine, theophylline, theobromine, and aminophylline).

5. Topical Anesthetics

The compositions of the present invention may also comprise a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

6. Skin Lightening Agents

The compositions of the present invention may comprise a skin lightening agent. Suitable skin lightening agents include those known in the art, including kojic acid, arbutin, tranexamic acid, ascorbic acid and derivatives, e.g., magnesium ascorbyl phosphate or sodium ascorbyl phosphate or other salts of ascorbyl phosphate.

7. Antimicrobial and Antifungal Actives

The compositions of the present invention may comprise an antimicrobial or antifungal active. Such actives are capable of destroying microbes, preventing the development of microbes or preventing the pathogenic action of microbes.

8. Sunscreen Actives

Generally, the compositions can comprise from about 0.5% to about 20% of the sunscreen actives. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

Also particularly useful in the compositions are sunscreen actives such as those disclosed in U.S. Pat. Nos. 4,937,370 and 4,999,186. The sunscreening agents disclosed therein have, in a single molecule, two distinct chromophore moieties, which exhibit different ultra-violet radiation absorption spectra. One of the chromophore moieties absorbs predominantly in the UVB radiation range and the other absorbs strongly in the UVA radiation range.

9. Conditioning Agents

The compositions of the present invention may comprise a conditioning agent selected from the group consisting of humectants, moisturizers, or skin conditioners. A variety of these materials can be employed and include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; and mixtures thereof. Also useful herein are the propoxylated glycerols and various C₁-C₃₀ monoesters and polyesters of sugars and related materials.

Preferably, the conditioning agent is selected from the group consisting of glycerol, urea, guanidine, sucrose polyester, and combinations thereof.

10. Thickening Agent

The compositions of the present invention can comprise one or more thickening agents.

(i) Carboxylic Acid Polymers

The compositions of the present invention can optionally comprise carboxylic acid polymers. These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. Examples of carboxylic acid polymer thickeners useful herein are those selected from the group consisting of carbomers, acrylates/C₁₀-C₃₀ alkyl acrylate crosspolymers, and mixtures thereof.

(ii) Crosslinked Polyacrylate Polymers

The compositions of the present invention can optionally comprise crosslinked polyacrylate polymers useful as thickeners or gelling agents including anionic, cationic and nonionic polymers, with the cationics being generally preferred.

(iii) Polyacrylamide Polymers

The compositions of the present invention can—optionally comprise polyacrylamide polymers, especially nonionic polyacrylamide polymers including substituted branched or unbranched polymers. Preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

(iv) Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage.

Other useful polysaccharides include scleroglucans comprising a linear chain of (1-3) linked glucose units with a (1-6) linked glucose every three units, a commercially available example of which is Clearogel™ CS11 from Michel Mercier Products Inc. (Mountainside, N.J.).

(v) Gums

Other thickening and gelling agents useful herein include materials that are primarily derived from natural sources. Nonlimiting examples of these gelling agent gums include materials selected from the group consisting of acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

### Method of Making

The compositions of the present invention are generally prepared by conventional methods such as are known in the art of making personal care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), and use of appropriate photostability approaches (e.g., incorporation of sunscreen/sunblock, use of opaque packaging).

### Methods of Use

The compositions of the present invention are useful for regulating keratinous tissue, particularly hair growth and mammalian skin condition. Such regulation of keratinous tissue conditions can include prophylactic and therapeutic regulation. It may also include providing a more noticeable improvement, both tactile and visual, in the appearance and feel of the hair on the skin of a mammal. Such methods provide ease, frequency, and effectiveness of shaving on a mammal, as slower re-growth of the hair allows treated skin to be shaved less frequently, thereby reducing irritation and erythema, and wounding events such as nicks and/or cuts. By slowing down the re-growth of the hair, the hair becomes less noticeable, softer, and/or finer and the skin is left feeling smoother and/or silkier. Additional benefits include improvements in the ease of shaving and increased shaving efficiency. Thus, the compositions of the present invention are useful in inhibiting hair growth, reducing shaving frequency, improving ease of shaving, decreasing shaving frequency, making hair softer and/or finer, making hair less noticeable, slowing the re-growth of hair, reducing erythema and irritation to skin, making skin smoother and/or silkier, and improving the hair removal process.

Examples of regulating skin conditions include, but are not limited to thickening keratinous tissue (i.e., building the epidermis and/or dermis layers of the skin and where applicable the keratinous layers of the nail and hair shaft) and preventing and/or retarding atrophy of mammalian skin, preventing and/or retarding the appearance of spider vessels and/or red blotchiness on mammalian skin, treating (i.e. preventing and/or retarding the appearance of) dark circles under the eye of a mammal, preventing and/or retarding sallowness of mammalian skin, regulating (i.e. preventing and/or retarding) sagging of mammalian skin, softening and/or smoothing lips, hair and nails of a mammal, preventing and/or relieving itch of mammalian skin, regulating skin texture (e.g. wrinkles and fine lines), regulating the appearance of shiny skin, treating (i.e. preventing and/or retarding the appearance of) cellulite, increasing the rate of skin turnover, and improving skin color (e.g. redness, freckles).

Regulating keratinous tissue condition is preferably practiced by applying a composition in the form of a skin lotion, cream, gel, foam, ointment, paste, serum, stick, emulsion, spray, conditioner, tonic, cosmetic, lipstick, foundation, nail polish, after-shave, or the like which is preferably intended to be left on the keratin structure for some esthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, still more preferably for at least several hours, e.g., up to about 12 hours. Any part of the external portion of the face, hair, and/or nails can be treated, e.g., face, lips, under-eye area, upper lip, eyelids, scalp, neck, torso, arms, underarms, hands, legs, feet, fingernails, toenails, scalp hair, eyelashes, eyebrows, etc. The composition can be applied with the fingers or with an implement or device (e.g., pad, cotton ball, applicator pen, spray applicator, and the like).

Another approach to ensure a continuous exposure of the skin to at least a minimum level of the skin care active is to apply the compound by use of a patch applied, e.g., to the face. Such an approach is particularly useful for problem skin areas needing more intensive treatment (e.g., facial crows feet area, frown lines, under eye area, upper lip and the like). The patch can be occlusive, semi-occlusive or non-occlusive and can be adhesive or non-adhesive. The composition can be contained within the patch or be applied to the skin prior to application of the patch. The patch can also include additional actives such as chemical initiators for exothermic reactions such as those described in U.S. Pat. Nos. 5,821,250, 5,981,547, and 5,972,957 to Wu, et al. The patch is preferably left on the skin for a period of at least about 5 minutes, more preferably at least about 15 minutes, more preferably still at least about 30 minutes, even more preferably at least about 1 hour, still more preferably at night as a form of night therapy.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application, are, in mg composition/cm² skin, from about 0.1 mg/cm² to about 20 mg/cm². A particularly useful application amount is about 0.5 mg/cm² to about 10 mg/cm².

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-2

Moisturizing Skin Cream/Lotion

Example

| | 1 | 2 |
|---|---|---|
| Component | % w/w | % w/w |
| Polyquaternium 37 | 1.0 | 1.0 |
| Green tea extract | 5.0 | 5.0 |
| Disodium EDTA | 0.1 | 0.1 |
| 1,2 Hexanediol | 3.0 | 3.0 |
| Glycerine | 7.0 | 7.0 |
| D-Panthenol | 1.0 | 1.0 |
| Sodium Hydroxide | 0.01 | 0.01 |
| Cetearyl Glucoside | 0.2 | 0.2 |
| Ethyl Paraben | 0.2 | 0.2 |
| Propyl Paraben | 0.1 | 0.1 |
| BHT | 1.0 | 0.5 |
| Stearyl alcohol | 0.6 | 0.64 |
| Menthyl anthranilate | 0 | 5 |
| Cetyl alcohol | 0.6 | 0.6 |
| Behenyl Alcohol | 0.4 | 0.4 |
| PEG100 Stearate | 0.1 | 0.1 |
| Polymethylsilsesquioxance | 1.0 | 1.0 |
| Isohexadecane | 3.0 | 3.0 |
| Isopropyl isostearate | 1.5 | 1.5 |
| Sucrose Polycottonseedate | 0.5 | 0.5 |
| DL-alpha Tocopheryl acetate | 0.3 | 0.3 |
| Petrolatum | 0.1 | 0.1 |
| Perfume | 0.3 | 0.3 |
| Dimethicone & Dimethiconol | 1.0 | 1.0 |
| Benzyl alcohol | 0.2 | 0.2 |
| Deionised Water | qs | qs |

### Examples 3-6

Moisturizing Skin Cream/Lotion

| | Example | | | |
|---|---|---|---|---|
| | 3 | 4 | 5 | 6 |
| Component | % w/w | % w/w | % w/w | % w/w |
| Niacinamide | 2.0 | 4.0 | 6.0 | 6.0 |
| Retinyl Propionate | 0.2 | 0.2 | 0.2 | 0.2 |
| Panthenol | 1.0 | 2.0 | 0.5 | 0.5 |
| Polyacrylamide & | 2.0 | 2.0 | 2.0 | 2.0 |
| isoparaffin & | | | | |
| laureth-7 | | | | |
| Glycerine | 7 | 7 | 7 | 7 |
| Hexanediol | 3 | 3 | 3 | 3 |
| Allantoin | 0.2 | 0.05 | 0.1 | 0.1 |
| Aloe vera gel | 0.1 | 0.1 | 0.1 | 0.1 |
| Tocopheryl acetate | 0.75 | 0.5 | 0.5 | 0.5 |
| Cetyl alcohol | 2.0 | 1.0 | 1.25 | 1.25 |
| Stearyl alcohol | 2.0 | 1.0 | 1.25 | 1.25 |
| Behenyl alcohol | 1.0 | 1.0 | 1.25 | 1.25 |
| Dimethicone & | 0.75 | 0.5 | 0.50 | 0.50 |
| dimethiconol | | | | |
| Steareth-21 | 0.6 | 0.4 | 0.5 | 0.5 |
| Steareth-2 | 0.1 | 0.08 | 0.03 | 0.03 |
| PPG-15 stearyl ether | 3.0 | 2.0 | 1.00 | 1.00 |
| Isohexadecane | 0 | 7.0 | 5.0 | 5.0 |
| Green tea extract | 5.0 | 5.0 | 5.0 | 5.0 |
| Menthyl anthranilate | 0 | 0 | 5.0 | 5.0 |
| Isononyl isononanoate | 5.0 | 0 | 0 | 5.0 |
| Dimethicone | 0.5 | 0.0 | 0.60 | 0.60 |
| (350 mm²s⁻¹) | | | | |
| Disodium EDTA | 0.10 | 0.10 | 0.10 | 0.10 |
| Nylon 12¹ | 1.5 | 1.0 | 1.1 | 1.1 |
| Titanium Dioxide (and) | 0.75 | 1.5 | 1.25 | 1.25 |
| Mica² | | | | |
| BHT | 1.00 | 1.00 | 1.00 | 1.00 |
| Petrolatum | 1.00 | 4.00 | 2.00 | 2.00 |
| Deionised water, | qs | qs | qs | qs |
| fragrance, | | | | |
| presevatives | | | | |

¹Orgasol ® 2002 D NAT COS.
²A green interference pigment

### Examples 7-11

Antiperspirant Soft Solid/Cream

| | Example | | | | |
|---|---|---|---|---|---|
| | 7 | 8 | 9 | 10 | 11 |
| Component | % w/w | % w/w | % w/w | % w/w | % w/w |
| Al Zr Trichlorohydrex | 25 | 25 | 25 | 25 | 25 |
| Glycinate (solid) | | | | | |
| Dimethicone (10cs) | 5.0 | 5.0 | 5.0 | 5.0 | 5.0 |
| Fully Hydrogenated | 5.0 | 5.0 | 5.0 | 5.0 | 5.0 |
| High Erucic Acid | | | | | |
| Rapeseed oil | | | | | |
| (HEAR oil) | | | | | |
| Green tea extract | 5.0 | 5.0 | 5.0 | 5.0 | 5.0 |
| Menthyl anthranilate | 5.0 | 5.0 | 5.0 | 5.0 | 5.0 |
| C-18-36 Acid | 1.25 | 1.25 | 1.25 | 1.25 | 1.25 |
| Triglyceride | | | | | |
| Syncrowax HGLC | | | | | |
| Perfume | 0.8 | 0.8 | 0.8 | 0.8 | 0.8 |
| Calcium Pantothenate | 0.5 | 0 | 3.5 | 0 | 0 |
| (solid) | | | | | |
| BHT | 0.5 | 0.5 | 0.5 | 0.5 | 0.5 |
| Tocopherol Acetate | 0.5 | 0 | 0.5 | 0.5 | 0 |
| Cyclopentasiloxane | qs | qs | qs | qs | qs |

### Example 12-13

Foundation Compact

Example

| | 12 | 13 |
|---|---|---|
| Component | % w/w | % w/w |
| TiO2 silicone treated (SAT treated | 5.0 | 5.0 |
| Tronox CR 837 supplied US Cosmetics) | | |
| Pigment | 1.2 | 1.2 |
| Talc (silicone treated) (Hydrophobic | 2.3 | 2.3 |
| Talc 9742 supplied by Warner Jenkinson) | | |
| Green tea extract | 5.0 | 5.0 |
| Menthyl anthranilate | 0 | 5 |
| TiO2 -MT100T (micronized TiO2 | 0.2 | 0.2 |
| supplied by Tri-K) | | |
| DC5225C (dimethicone copolyol - | 0.3 | 0.3 |
| 10% active in cyclomethicone) | | |
| GE SFE 839 Cross-linked Siloxane | 48 | 48 |
| Elastomer Gel¹ | | |
| Propylparaben (preservative) | 0.10 | 0.10 |
| BHT | 0.5 | 0.5 |
| Glycerine | 7.0 | 7.0 |
| Ozokerite Wax | 3.2 | 3.2 |
| DC245 (cyclomethicone) | qs | qs |

¹5% Dimethicone/vinyl dimethicone cross-polymer in cyclomethicone

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

1. A personal care composition comprising
a) at least one chronic skin care active selected from the group consisting of butylated hydroxytoluene, butylated hydroxyanisole, hexyl isobutyrate, menthyl anthranilate, methofuran, 3-butylidenepthalide, cetyl pyridinium chloride, green tea extract, catechins, phytosterols, ursolic acid, and plant extract compounds;
b) at least one acute skin care active selected from the group consisting of particulate materials, alkane polyols, panthenol, pantothenic acid derivatives, and tanning actives; and
c) a dermatologically acceptable carrier.

2. The composition of claim 1 wherein said particulate materials are selected from the group consisting of particles, pigments, and cross-linked silicone elastomers.

3. The composition of claim 1 wherein said composition further comprises at least one additional component selected from the group consisting of hair growth inhibiting compounds, polyquaternium 37, depilatories, desquamation actives, anti-acne actives, anti-wrinkle actives, anti-atrophy actives, anti-oxidant actives, radical scavengers, chelators, anti-inflammatory agents, anti-cellulite agents, topical anesthetics, skin lightening agents, antimicrobial and antifungal actives, sunscreen actives, conditioning agents, thickening agents, and mixtures thereof.

4. The composition of claim 1 wherein said composition further comprises polyquaternium 37.

5. The composition of claim 1 wherein said chronic skin care active comprises cetyl pyridinium chloride and said composition further comprises polyquaternium 37.

6. A method of reducing shaving frequency, said method comprising the step of topically applying to the skin or hair of a mammal the composition according to claim 1.

7. A method of inhibiting mammalian hair growth, said method comprising the step of topically applying to the skin or hair of a mammal the composition according to claim 1.

8. A method of improving ease of shaving, said method comprising the step of topically applying to the skin or hair of a mammal the composition according to claim 1.

9. A method of increasing shaving efficiency, said method comprising the step of topically applying to the skin or hair of a mammal the composition according to claim 1.

10. A method of making hair softer and finer, said method comprising the step of topically applying to the skin or hair of a mammal the composition according to claim 1.

11. A method of making hair less noticeable, said method comprising the step of topically applying to the skin or hair of a mammal the composition according to claim 1.

12. A method slowing the re-growth of hair, said method comprising the step of topically applying to the skin or hair of a mammal the composition according to claim 1.

13. A method of reducing erythema and irritation to skin, said method comprising the step of topically applying to the skin or hair of a mammal the composition according to claim 1.

14. A method of making skin smoother and silkier, said method comprising the step of topically applying to the skin or hair of a mammal the composition according to claim 1.

15. A method of improving the hair removal process, said method comprising the step of topically applying to the skin or hair of a mammal the composition according to claim 1.

16. A method of inhibiting mammalian hair growth, said method comprising the step of topically applying a safe and effective amount of a compound selected from the group consisting of menthyl anthranilate, its salts, its derivatives, and mixtures thereof, and a dermatologically acceptable carrier to the skin of a mammal in need of treatment.

17. A method of inhibiting mammalian hair growth, said method comprising the step of topically applying a safe and effective amount of a compound selected from the group consisting of hexyl isobutyrate, its salts, its derivatives, and mixtures thereof, and a dermatologically acceptable carrier to the skin of a mammal in need of treatment.

18. A method of inhibiting mammalian hair growth, said method comprising the step of topically applying a safe and effective amount of a compound selected from the group consisting of methofuran, its salts, its derivatives, and mixtures thereof; and a dermatologically acceptable carrier to the skin of a mammal in need of treatment.

19. A method of inhibiting mammalian hair growth, said method comprising the step of topically applying a safe and effective amount of a compound selected from the group consisting of 3-butylidenepthalide, its salts, its derivatives, and mixtures thereof; and a dermatologically acceptable carrier to the skin of a mammal in need of treatment.
